(19) United States
(12) Patent Application Publication
(10) Pub. No.: US 2022/0226695 A1
(43) Pub. Date: Jul. 21, 2022

(54) SYSTEMS AND METHODS FOR WORKOUT TRACKING AND CLASSIFICATION

(71) Applicant: CoPilot Systems Inc., Pittsburgh, PA (US)

(72) Inventors: Gabriel Madonna, Pittsburgh, PA (US); Matthew Spettel, Pittsburgh, PA (US)

(73) Assignee: CoPilot Systems Inc., Pittsburgh, PA (US)

(21) Appl. No.: 17/614,611

(22) PCT Filed: May 28, 2020
(86) PCT No.: PCT/US2020/034877

§ 371 Date: Nov. 29, 2021

Related U.S. Application Data
(60) Provisional application No. 62/853,482, filed on May 28, 2019.

(51) Int. Cl. A63B 24/00

(52) U.S. Cl. CPC A63B 24/0062; A63B 24/0006; A63B 2024/0068; A63B 2024/0009; A63B 2220/14; A63B 2230/01; A63B 2220/62; A63B 2220/803; A63B 2220/805; A63B 2225/54; A63B 2220/17

(57) ABSTRACT

Disclosed herein are various systems and methods for tracking and improving workout. As disclosed herein, exercise data associated with a user is received from a user device. Based on various other characteristics disclosed herein, the system may identify one or more known exercises from within the exercise data and then analyze the data related to the one or more known exercises to determine workout metrics. In some embodiments, the system may further evaluate the workout metrics using various techniques disclosed herein, and based on the evaluation a label may be associated with the workout metrics. The label and/or workout metrics may then be saved and/or provided to a user or another individual, such as a personal trainer.

### PRIORITY

This patent application claims priority to U.S. Provisional Patent Application No. 62/853,482, entitled “SYSTEMS AND METHODS FOR WORKOUT TRACKING AND CLASSIFICATION,” filed on May 28, 2019, which is hereby incorporated by reference in its entirety.

### BACKGROUND

The benefits of exercise are well understood; however, most people do not engage in sufficient physical activity. The reasons are varied, but include individual metabolism, work schedule, work type (e.g., labor v. stationary), age, etc. An additional impediment is that individuals have great difficulty in tracking their effort and progress over time. Although personal goals (e.g., physical ability, muscle mass, weight loss, increased endurance, or just looking better at the beach) may vary among individuals, maintaining the drive and effort required to workout is difficult over the long term even if discernible progress has been made.

More accurate tracking of progress may provide substantial benefits for weight trainers and/or the people training them. For example, if an individual is able to track not only their progress, but also which exercises lead to such progress, the individual may be able to accelerate their progress through such correlation and/or their trainer may be able to better hold them accountable or monitor their progress.

Tracking workouts has led to the creation of a multitude of workout logging applications for smartphones and other portable devices. Some of these applications merely encompass data entry. Others utilize information collected from wearable devices, such as smart watches, smart phones, etc. However, all such applications suffer from an inability to automatically record data without interrupting a workout. Even if an individual uses a system that records step count or a general activity level, the individual is still required to input specific activity details. For example, when lifting weights, conventional applications require a user to log specific details regarding the type of exercise being performed to have any significant value to the user post-exercising. Thus, each time a user finishes a set (i.e., a predefined set of exercise repetitions), the user must record (e.g., on their electronic device or on paper) the type of exercise, the number of repetitions, etc.

Accordingly, a system is needed that can automate the data collection associated with weight lifting and other various physical activities.

### BRIEF DESCRIPTION OF THE DRAWINGS

For the purpose of illustrating the invention, there is shown in the drawings various embodiments. However, it should be understood, that the invention is not limited to the specific instrumentalities disclosed herein as they are used for illustrative purposes only. Included in the drawings are the following Figures:

FIG. 1 depicts an illustrative method for detecting exercise periods from raw inertial data over time in accordance with an embodiment.

FIG. 2 depicts an illustrative method for determining rep intervals in an exercise period in accordance with an embodiment.

FIG. 3 depicts an illustrative method for classifying an exercise based on a statistical analysis of the collection of one more reps in accordance with an embodiment.

FIG. 4 depicts an illustrative method for using dynamic time warping to compare a single rep with the content of a training set in accordance with an embodiment.

FIG. 5 depicts an example graph for an assessment regarding the classification of a time period belonging to an exercise period in accordance with an embodiment.

FIG. 6. depicts an illustrative example of a use case summary in accordance with an embodiment.

FIG. 7 depicts an illustrative computer system for analyzing and classifying various physical activities in accordance with an embodiment.

### DETAILED DESCRIPTION

This disclosure is not limited to the particular systems, devices, and methods described, as these may vary. The terminology used in the description is for the purpose of describing the particular versions or embodiments only, and is not intended to limit the scope

As used in this document, the singular forms “a,” “an,” and “the” include plural references unless the context clearly dictates otherwise. Moreover, the present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As discussed herein, a system and/or method are needed to improve automated data collection during exercise. In some embodiments, an information handling device (e.g., a smart phone, laptop, smart watch, PC, server, etc.) may receive movement data from one or more sensors (e.g., a local sensor or a sensor on a remote device). The information handling device may analyze the movement data to identify one or more periodic windows within the movement data. The one or more periodic windows may be further analyzed to determine whether such windows relate to a training set of a known exercise type. A known exercise may be any known or newly created exercise type, such as, for example, Arnold press, back extension, banana hold, barbell curl, barbell lunge, bench press, bent knee chair leg raise, bent over row, bicycle, bike, bodyweight lunge, bodyweight squat, box jump, bridge stretch, Bulgarian split squat, burpee, butterfly stretch, cable fly, cable row, calf roller, calf stretch, chair leg raise, chest opener, chest press, chin up, clam shell, clean and jerk, close grip bench press, crunch, dead bug, deadlift, decline bench press, decline dumbbell bench press, dip, dumbbell back extension, dumbbell bench press, dumbbell clean and press, dumbbell curl, dumbbell fly, dumbbell free shoulder press, dumbbell front raise, dumbbell hammer curl, dumbbell incline bench press, dumbbell lateral lunge, dumbbell lunge, dumbbell lying curl, dumbbell one leg deadlift, dumbbell overhead lunge, dumbbell pullover, dumbbell reverse fly, dumbbell reverse lunge, dumbbell row, dumbbell shoulder press, dumbbell shrug, dumbbell snatch, elliptical, extended dog stretch, extended plank, face pull, figure four stretch, flutter kick, front squat, glute march, glute roller, goblet squat, good morning, hammer lat pulldown, hamstring roller, hamstring stretch, hang clean, hang snatch, high knees, hip abduction, hip adduction, hip flexor stretch, hip thrust, it band roller, inchworm, incline bench press, javelin stretch, jump lunge, jump rope, jump squat, jumping jack, kettlebell swing, knee to chest stretch, lat pulldown, lat roller, lat stretch, lateral raise, leg cross body stretch, leg curl, leg extension, leg press, leg raise, lower back roller, lying hamstring stretch, lying toe touch, machine fly, machine shoulder press, med ball throw, mountain climber, no push up burpee, one arm lat pulldown, one leg deadlift, one leg leg press, overhead med ball slam, overhead press, plank, plank jack, plank reach, plank reach through, plank shoulder tap, plank to push up, pull up, push press, push up, push up to reach, quad roller, quad stretch, Romanian deadlift, rope tricep pushdown, row, run, Russian twist, seated straddle stretch, shoulder stretch, side bend, side med ball slam, side plank, sit up, skater jump, snatch, sphinx stretch, Spiderman, Spiderman stretch, spine rotation stretch, split stretch, sprinter lunge, squat, stairs, standing jump, step up, straddle stretch, superman, trx chest press, trx hollow body, trx one arm row, trx pistol squat, trx reverse fly, trx row, thruster, toe touch crunch, tricep extension, tricep stretch, trunk rotation, two leg hamstring stretch, upper back roller, v up, walk, wall handstand, wall sit, weighted jump, weighted jump lunge, weighted Russian twist, wide cable row, windshield wiper, or the like. It should be noted that this the above list is non-limiting and is intended for illustrative purposes only. Accordingly, one or more embodiments may detect and/or identify exercises not listed herein.

It should be understood that the one or more remote devices may be any type of current or newly developed tracking device technology, such as smart phones, wearable devices, image trackers, etc. Accordingly, although wearable devices are discussed herein as the primary embodiment, it is only for illustrative purposes, and thus not intended to be limiting in nature. Additionally, although reference is made herein to a secondary device (e.g., a smart phone, PC, etc.), which receives and analyzes the data collected by the tracking device, it should be understood that various alternative embodiments may exist in which the tracking device (e.g., smart watch, wearable device, etc.) is both collecting and analyzing the data.

Accordingly, in some embodiments, a user may wear one or more wearable devices (e.g., a smart watch, a Fitbit® tracker, a smart phone, a wearable device, etc.) that includes an inertial measurement unit (IMU). The wearable device may track the user's training session (e.g., in real time). In a further embodiment, the system (e.g., tracking device, computational device, transmission device, etc.) may automatically detect when a user is performing an exercise (i.e., training set, rep, etc.). As discussed further herein, information pertaining to the training set may be further analyzed and/or processed to isolate each repetition (rep) within a set. Once each rep is isolated and identified, the total number of reps in a set or workout may be counted or tallied.

In a further embodiment, each rep and/or training set may be classified based on the type of exercise (e.g., pushup, bench press, bicep curl, etc.). Additionally or alternatively, an exercise may be known to the system before the set starts, and such information may be used to increase the effectiveness of the pipeline. Accordingly, a user will rarely, if ever, need to manually interact with the system (e.g., entering exercise information). In some embodiments, the user may be prompted (e.g., a notification may be provided on their smartphone or smart wearable device) to enter, correct, and/or modify the collected data related to the most recent workout. For example, the system may request that the user enter additional information, such as, for example, the amount of weight lifted in each exercise or training set or the type of exercise equipment used (e.g., free-weights, resistance bands, etc.).

As discussed herein, in some embodiments, the system may use multiple processing devices to record, analyze, and present the data. Thus, in some embodiments, data collected from the IMU(s) may be packaged and/or compressed and sent to a remote device for processing.

In some embodiments, continuous data transfer may be required. In such embodiments requiring continuous data transfer, the system may transfer the IMU data to an information handling device, which monitors/samples the collection frequency of the IMU data. By monitoring the collection frequency, the system may, in a further embodiment, identify if the collection frequency is under a particular threshold (e.g., approximately 10 Hz), which may cause degradation or loss of the captured data. As such, in some embodiments, if the system determines that the collection frequency is less than a threshold frequency, a notification (e.g., audio, visual, haptic, etc.) may be sent to the user (e.g., via a wearable device, smartphone, computer, etc.) informing them of the low sampling rate and/or suggesting potential solutions to improve the connection and/or the sampling rate. In other embodiments, the collection frequency may be maintained between two specific thresholds (e.g., above 15 Hz and below 100 Hz). In order to reduce or eliminate the degradation/loss of the IMU data. In some embodiments, the data from the IMU may be cached in one or more storage locations prior to transfer and/or processing to reduce potential errors.

In some embodiments, the device with the IMU(s) (e.g., the wearable device) may have one or more storage caches to store the IMU data before, during, and after transfer. In an additional or alternative embodiment, the remote processor (e.g., system server) or an intermediate device (e.g., a smartphone) may have one or more storage caches to store the IMU data as the data is received. Based on the above, in some embodiments, one or more processing devices (e.g., a wearable device, a smartphone device, a server device, etc.) may have one or more storage caches to maintain the IMU data. In some embodiments, maintaining the IMU data may allow a processing device to track and/or detect if the collection frequency is above a threshold or between an upper threshold and a lower threshold.

As discussed herein, in some embodiments, the data processing may occur at a remote server as opposed to being performed on the wearable device. In such embodiments, it may be beneficial to compress the raw IMU data before, during, and/or after data transfers. For example, in some embodiments, the floating point measurements output by the IMU may be mapped to single-byte integers. In some embodiments, this mapping function may employ a supervised machine learning algorithm. In a further embodiment, the machine learning algorithm may have been trained (or is continuously trained) using a collection of aggregated data (e.g., historical aggregated data, newly aggregated data, real-time aggregated data, etc.). In further embodiments, the mapping function may be a linear transformation. Additional metadata may be packaged with the compressed IMU data comprising a user ID, an expected number of reps, a weight, a set ID, or a workout ID. In embodiments using an external data processing server, communication between the user and the server may be via Web Sockets, HTTP, or any other communications protocol.

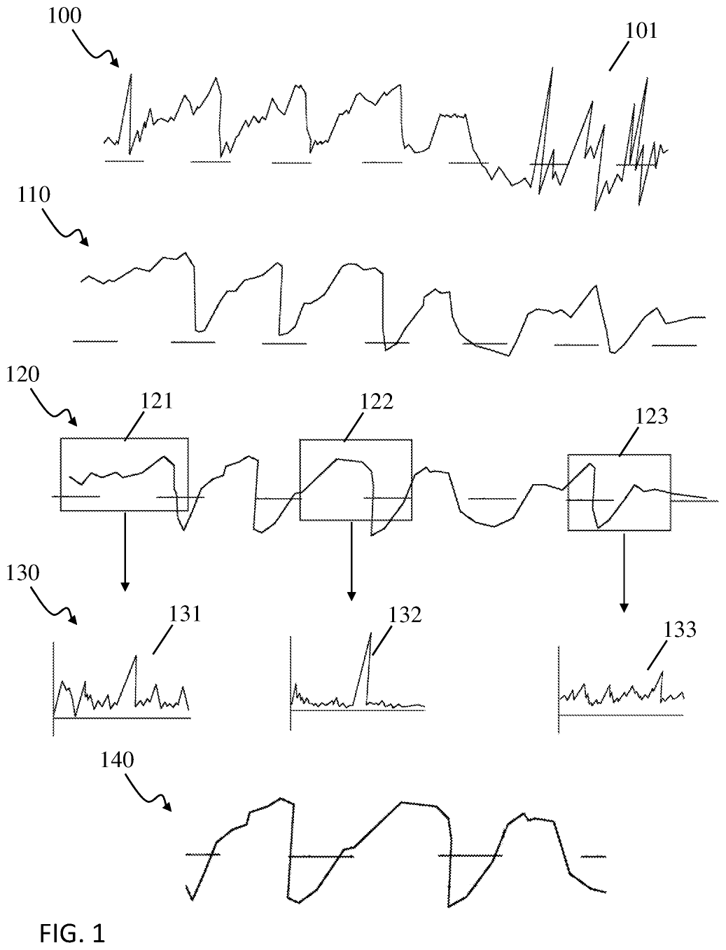

Referring now to FIG. 1, various graphs and waveforms are shown according to an embodiment. As discussed herein, biometric data 100 (e.g., movement data, heart rate data, preparation data, muscle contraction data, etc.) may be captured and/or received using an information handling device. In some embodiments, such as the embodiment shown in FIG. 1, the biometric data 100 may include noisy or non-uniform sections 101 that make data analytics more difficult. Accordingly, in some embodiments, the biometric data 100 may be processed (e.g., smoothed and/or normalized) 110 to correct for various environmental or mechanical factors (e.g., gravity, wind, poor device calibration, device error, device interference, etc.). The normalized or smoothed data 110/120 may be analyzed to identify one or more windows (e.g., 121, 122, and 123). In some embodiments, each window may be vectorized.

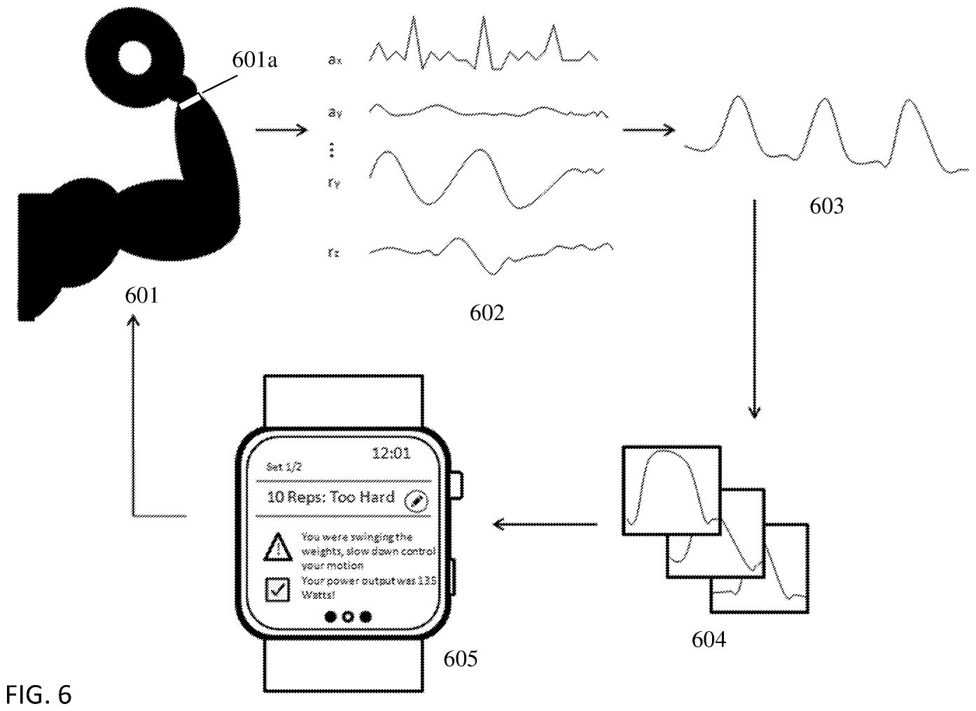

The vector data for each window may include various metrics. For example, when a user performs an exercise, the user's movements may be collected, using the various system/methods described herein, as a packet. In some embodiments, the collected packet may comprise gravity signals for the x, y, and z axes. In a further embodiment, the collected packet may comprise acceleration metrics, rotation metrics, and velocity metrics. Referring briefly to FIG. 6, in some embodiments, the packet 602 (and the various metrics) are analyzed and generated/synthesized into one or more components (e.g., a magnitude component, a first principle component in all three gravity columns, a first principle component of the y and z, gravity columns, acceleration, rotation, etc.). Using the generated components, the system may, in some embodiments, extract various features from the data.

A non-limiting illustrative example of potential features for extraction may include: a “feature vector” feature representing the time at the center of the window, an “auto max” feature representing the maximum value of the auto correlation, an “auto peak” feature representing the number of peaks in the autocorrelation, a power value feature at various spectrums (e.g., 1 Hz, 5 Hz, 10 Hz, 20 Hz, etc.), a “mean” feature representing the mean value of the column over that window, a “std” feature representing a standard deviation of the column over that window, a “RMS” feature representing a root mean square of the column over that window, etc. Those of ordinary skill in the art will understand that the above features merely provide a representative sample of all potential features and that 10, 20, 30, 40, 50 or more features may be extracted. Once the extracted feature set is complete, the system may, in some embodiments, combine the feature set into a single signal, such as that shown in FIG. 6 at 603.

In another embodiment, an information handling device may perform a frequency analysis (e.g., a Fourier transform) or one or more alternative forms of feature extraction (e.g., pca, mean, median, std, variance, RMS, autocorrelation, etc.) on the selected data (i.e., the windowed data) 121, 122, and 123. The frequency analysis 130 may be used to determine a periodicity of the windowed portion. As would be understood by one of ordinary skill in the art, training sets generally have inherently strong periodicity due to the repetitive motion of each set. Accordingly, in some embodiments, each windowed portion may be mapped to a periodogram or graphed according to its spectral density 131, 132, and 133.

Additionally, certain exercises may belong in, or be associated with, a certain environment and/or feature space which is distinct from the environment and/or feature space occupied when resting (e.g., when performing pull-ups, the average value of the direction of an individual's arms will correspond to having the individual's arms above the individual's head as opposed to pointing at the ground). This can be used in conjunction with or in place of frequency-based feature extraction, such as periodograms.

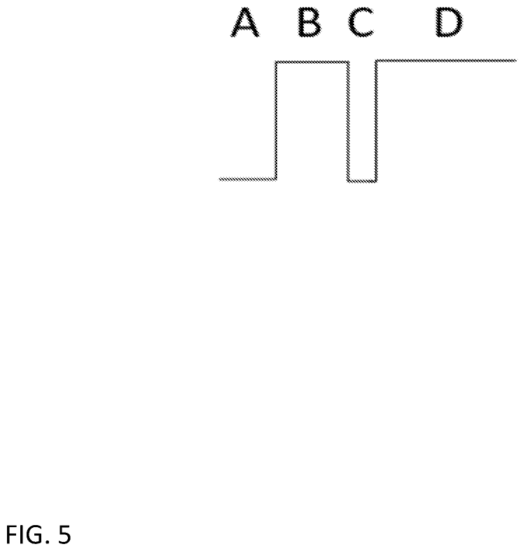

As shown, the analysis of window 122 may identify a single large spike in frequency 132 from which the system may infer that the user was likely exercising at this time. Similarly, the analysis of windows 121 and 123 may identify that such windows contain more noise and no discernable peak. Accordingly, it is likely that window 121 corresponds to a time when the exercise was first beginning and window 123 corresponds to a time when the exercise was concluding. In a further embodiment, an artificial neural network (e.g., a deep neural network (DNN), a deep belief network (DBN), a recurrent neural network (RNN), a convolutional neural network (CNN), an auto-encoder, and a deep learning system (DLS)) or some other model (e.g., a support vector machine (SVM), a logistic regression, random forests, etc.) may be used to analyze the feature vector and provide a binary classification or regression (see FIG. 5 for an illustrative example) denoting whether each window belongs to an exercise period.

In some embodiments, error correction or classification buffering may be required. Accordingly, as discussed herein, the output of a binary classifier may be modified to remove outliers or potential errors as a means of error correction. In some embodiments, this may be accomplished by examining the binary output in the context of one or more neighboring outputs. Referring briefly to FIG. 5, an embodiment is shown that depicts a graph of four adjacent time frame windows, A, B, C, and D. As shown, windows A and C returned a false outcome (i.e., binary 0) while B and D returned a true outcome (i.e., binary 1). Because time frame C is sufficiently short and is located between two true outcomes, the system may alter its outcome (i.e., to a binary 1 or true status) based on an assumption that a user could not physically stop and restart exercising in time period less than a threshold. Additionally or alternatively, the system may alter the values for isolated true windows to false under the assumption that a set would not last for a time period less than a threshold. In a further embodiment, a classification buffer may operate similar to an accumulator, which saturates with the accumulation of confidence that recent packets have represented “in set” periods and which depletes when recent packets have represented “rest” periods.

Referring back to FIG. 1, once a number of periodic windows have been identified and analyzed, an information handling device may identify a continuous block of windows that each returned true, or binary 1. Such windows may be linked and/or associated with each other to create a complete exercise set 140.

Those of ordinary skill in the art will recognize that modifications could be made to the operations performed herein. For example, if the received biometric data 100 exhibits a viable feature vector, the biometric data may not be smoothed and/or normalized 110 prior to the window creation 120 and/or frequency analysis 130. Alternatively, the frequency analysis 130 may result in a different feature vector than the periodogram, such as one based on variance (not shown). In a further embodiment, one or more of the operations may be replaced with a trained neural network, which may generate a representative state vector. In additional embodiments, other systems, such as a convolutional neural network or recurrent neural network, may be used. Alternatively, a stochastic block model may be employed. Furthermore, these one or more steps may be performed by thresholding the values of the periodogram and/or using a support vector machine.

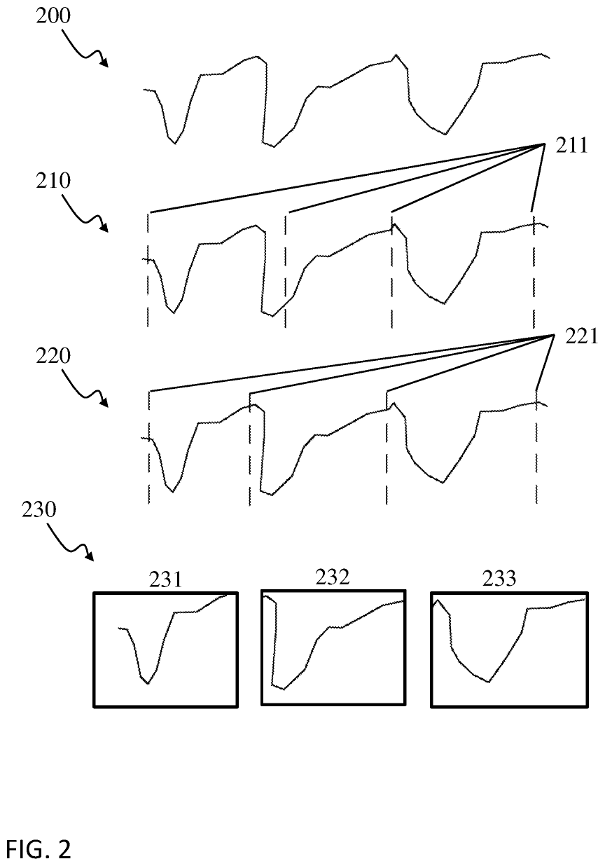

Referring now to FIG. 2, once a completed exercise set 200 (140 of FIG. 1) is identified, a system may initiate a rep detection process. As discussed herein, rep detection may be performed after a complete set has been parsed out. In an alternate embodiment, rep detection may be performed in real time or substantially in real time by analyzing identified portions of the biometric data that comprise one or more windows, and are thus indicated as being part of a training set. Accordingly, in some embodiments, the system may operate in an unsupervised fashion. Moreover, in some embodiments, the system may be highly scalable because it only relies on an assumption that each rep is, or will be, structurally similar to the most adjacent rep. Accordingly, in some embodiments, the system may use, for example and without limitation, historical information, statistical analysis, and/or a random number generator to determine the indices 211 demarcating reps in the set 200/210.

In a further embodiment, the determination of the indices 211 demarcating reps may be based at least in part on the periodicity identified during a frequency analysis operation. Thus, each index 211 may be modified (e.g., perturbed) by a decreasing, or an increasing, amount over a plurality of iterations. In some embodiments, a similarity score may be calculated between each rep and each of the reps that are immediately before and after such rep (e.g., reps that share an index line 211). In a further embodiment, the information handling device may modify the location of the indices, such as, for example, from 211 to 221. This modification and recalculation may be carried out multiple times for each index 211/221 in order to create the most similar periodic pattern 230.

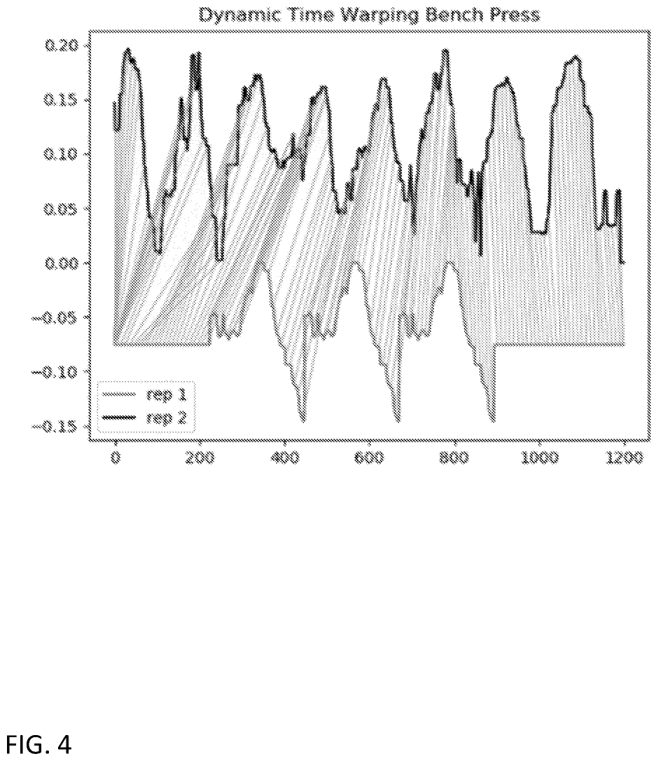

In some embodiments, each pair of reps within a set may be compared, and a similarity measure may be determined. The similarity measure may be determined via, for example and without limitation, correlation, dynamic time warping (DTW), and/or any other signal comparison metric. By way of non-limiting example, dynamic time warping between a first rep and a plurality of other reps of a full exercise set is depicted in FIG. 4. In some embodiments, a score may be normalized to fall within a standardized range. In some embodiments, a score may be biased to account for concepts not otherwise addressed by this metric (e.g., to assume that reps should be the same length). Returning briefly to FIG. 2, the optimized similar reps 231, 232, and 233 may be parsed or isolated from the biometric data in order to determine the number of reps, and/or to be further analyzed in order to identify an exercise type.

In another embodiment, a repository approach may be used for rep detection. For example, an information handling device may access a repository, or database, that contains one or more stored rep templates (e.g., one or more rep templates for each exercise type). In some embodiments, the templates may be compared to one or more training sets, similar to the scoring discussed herein. For example, an illustrative system may utilize dynamic time warping to generate a mapping scheme from one or more points in a template to one or more points in the set. Such a mapping scheme may be possible because the one or more points in the template are time invariant. In other words, if the user performs reps which take half the time of a known rep (e.g., a template rep), the beginning of the template rep may be mapped to the beginning of the user's rep and the end of the template may be mapped to the end of the user's rep without consideration of the amount of time required to perform the rep. A non-limiting example of this principle is shown in FIG. 4, in which the template with the highest similarity is used to parse out each additional rep. Various operations of the described process may be removed, modified, or repeated when identifying the periodic window of the exercise and the exercise type based on its relationship to the known template. Because each exercise generally has a unique motion curve, an exercise performed by a user can be associated with a known exercise based on a template for such exercise. The period for each rep may be determined based at least in part on the template.

In another embodiment, each of the various sensors on a user device (e.g., a wearable device, smart device, etc.) may have a peak and/or threshold metric. In some embodiments, the peak/threshold metric may be exceeded by one or more of the sensors. Based on exceeding the threshold of the one or more sensors, the system may be able to determine a demarcation (e.g., of a rep, set, workout, etc.).

In some embodiments, a similar strategy may be performed using other information (e.g., known, detected, etc.) about the set. For example, a peak or threshold analysis may be performed to determine an exercise that is being performed or to enable the system to determine a demarcation (e.g., of a rep, set, workout, etc.). In another embodiment, the other information (e.g., known, detected, etc.) may be used to inform the parameters of the peak detection (e.g., thresholds, smoothing, values of coefficients of combination if such a combination is used, etc.).

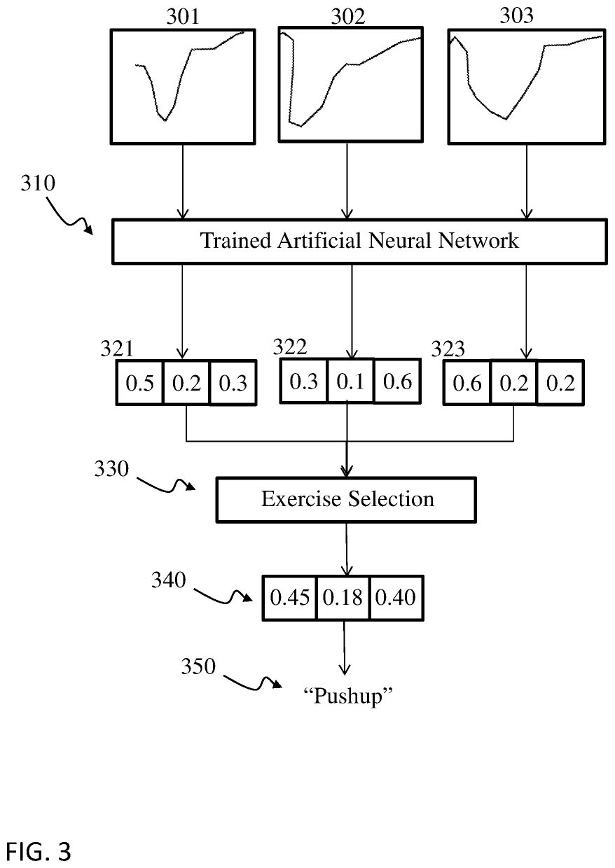

Referring now to FIG. 3, an example of exercise classification is shown according to an embodiment. In some embodiments, and as shown in FIG. 3, the parsed reps 301, 302, and 303 may be provided to a trained artificial neural network 310. The artificial neural network 310 may generate a vector of probabilities, e.g., 321, 322, and 323, for each of the parsed reps. In some embodiments, each probability in the vector of probabilities may correspond to a likelihood that the waveform is or was generated by a user performing a particular exercise. In a further embodiment, an exercise may be selected 330 as the exercise that is or was being performed. In an embodiment, the exercise may be selected 330 by, for example and without limitation, combining 340 the vectors of probabilities, and selecting the most probable exercise 350.

Additionally or alternatively, a different model may be used to generate the vector of probabilities. For example, the model may include a different type of neural network, a k-nearest neighbor model, a hidden Markov model, or the like. Accordingly, in some embodiments, the process of combining vectors may also be modified based on, for example and without limitation, the average, the argmax of the sum of the vectors, or the like. Moreover, in some embodiments, a neural network may be employed for this classification. The neural network may be used in tandem with a process of matching using a repository, as discussed herein, where the two results are combined. In a further embodiment, the data in the repository may be used to train a classification neural network.

Referring to FIG. 6, a use case summary is illustrated according to an embodiment. As shown in FIG. 6, a user 601 may perform a repetitive exercise while wearing an IMU 601a. The motion of the user may be sampled over periods of time in three dimensions (x, y, z) 602. The motion may be encoded into one signal 603. The reps may be demarcated according to one or more of the methods described herein 604. Feedback may then be generated and displayed to the user 605. In this embodiment, the display is on the user's smart watch, but the information may be displayed on any computer or mobile device. This feedback may then prompt a change in motion (e.g., form, rep rate, or exercise type) in the user 601.

In some embodiments, the system may be able to identify various factors of the exercise (e.g., explosiveness, time under tension, acceleration, velocity, rest period, form, quality/class, difficulty, user fatigue, etc.). In a further embodiment, the system may calculate an “effectiveness” level or value based on the identified factors. By way of non-limiting example, if a user performed a bicep curl or a set of bicep curls too quickly, the accelerations would likely be very high, and the time under tension would likely be very low. Isolation exercises, like a bicep curl, should generally have low acceleration (i.e., no swinging, rocking, etc.) and high time under tension.

Thus, in some embodiments, the system may be able to provide additional user feedback related to the effectiveness, difficultly, form, etc. of an exercise. For example, in some embodiments, the system may indicate a percent of one repetition max, a number of repetitions remaining before failure, a rate of perceived exertion, a predefined set of exercise-specific form types, a numerical rating, or a descriptive identifier (e.g., very easy, easy, neutral, difficult, very difficult, any value on a spectrum from extremely easy to extremely hard, a numerical rating, excellent, very good, good, fair, poor, very bad, any value on a spectrum from extremely good to extremely bad, etc.)

In some embodiments, the system may assign various labels (e.g., form labels, difficulty labels, and the like) based on the received user data (e.g., workout metrics, set-based exercise data, etc.). In a further embodiment, the system may assign a predefined set of exercise-specific form types, such as, for example, correct, swinging, bouncing, half, quarter, failed, inadequate, leaning, flaring elbows, lop-sided, kneeling, a numerical rating, or any term or description that would be understood by someone of ordinary skill in the art.

Because most exercise types have different “optimal” levels for each of the identified factors (e.g., acceleration, explosiveness, time under tension, etc.), the system can compare a given rep set against previously recorded rep sets to determine whether the given rep set differs in a statistically significant way from an optimal set. In a further embodiment, the system, or a user, may identify an “optimal” rep set. For example, a user may create an optimal set based on their preferences and/or a user could select/identify a specific rep set (e.g., a rep set done with trainer supervision) as an “optimal” rep set. Additionally or alternatively, the system may identify (e.g., using AI, machine learning, or any artificial neural network as discussed herein) one or more characteristics for an optimal rep set based on historical data, such as, for example, user specific historical data and/or historical data of the general population.

In further embodiments, metadata can be extracted from the collected set data to calculate other useful metrics. Example calculated metrics comprise a difficulty of performing a single rep, a quality of form for a rep, the form “class” of a rep/set (e.g., “swinging,” “leaning,” “good,” etc.), and an endurance function over multiple reps, These metrics may be used by a real or virtual trainer to optimize the training of a user and/or by the user to be more informed about their own performance These metrics may be used to calculate an optimal number of reps to be performed for a given weight value, to identify a weight to be used for a given number of reps, to inform the user/trainer of the user's form quality, and/or to help determine the user's progress.

In some embodiments, the user may be pushed to increase the number of reps that they perform until they meet a threshold of difficulty in performing an additional rep. This difficulty value may be averaged over multiple reps. The threshold value may be modified to maximize gains or minimize injury and recovery periods.

In further embodiments, a repository of user exercise data over time may be utilized. The user data found within may be anonymized In some embodiments, the metrics of a current user may be matched with past metrics of other users from the database. In some embodiments, the past users may represent a set of users that had positive gains in performance over time. Information corresponding to a stored user having a similar set of metrics may be used to generate a predictive model for the current user.

As one skilled in the art may see, the systems and methods disclosed herein are applicable outside of the space of traditional exercising. Specifically, the metrics may be useful in physical therapy. For example, a physical therapist may use historical data to better track and formulate a patient's recovery. Furthermore, a physical therapist may directly monitor remote, out of office, patient exercise, thereby allowing for faster, more affordable, and better guided care. In addition to physical therapy, the systems and methods disclosed herein may be applied to any activity with repeated human motion. Non-limiting examples may include analyzing movements of a user in a sport, such as monitoring a user's golf swing or throwing motion, and monitor a user's movement at work, such as warehouse worker that lifts certain items repetitively. The results of the analysis may lead to recommendations for improved form and/or downtime scheduling (e.g., breaks, stretches, or exercises).

Computer readable program instructions for carrying out operations disclosed herein may include assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++, or the like, and conventional procedural programming languages, such as “C,” Python, Swift, or similar programming languages. The computer readable program instructions may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network, a wide area network, and/or a metropolitan area network. In some embodiments, a connection may be made to an external computer (for example, through the Internet using an Internet Service Provider).

Aspects of the present invention are described herein with reference to flowchart illustrations and/or block diagrams of methods, apparatuses (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

Computer readable program instructions may be provided to a processor of a computer or other programmable data processing apparatus to produce a machine, such that the instructions, which execute via the processor of the computer or other programmable data processing apparatus, implement the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations to be performed on the computer, other programmable apparatus, or other device. The performance of such operations may produce a computer-implemented process, such that the instructions which are executed on the computer, other programmable apparatus, or other device implement the functions/acts specified in the flowchart and/or block diagram block or blocks.

The flowchart and block diagrams in the figures illustrate the architecture, functionality, and operation of potential implementations of systems, methods, and computer program products according to various embodiments. In this regard, each block in the flowchart or block diagrams may represent a module, segment, or portion of instructions, which comprises one or more executable instructions for implementing the specified logical functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

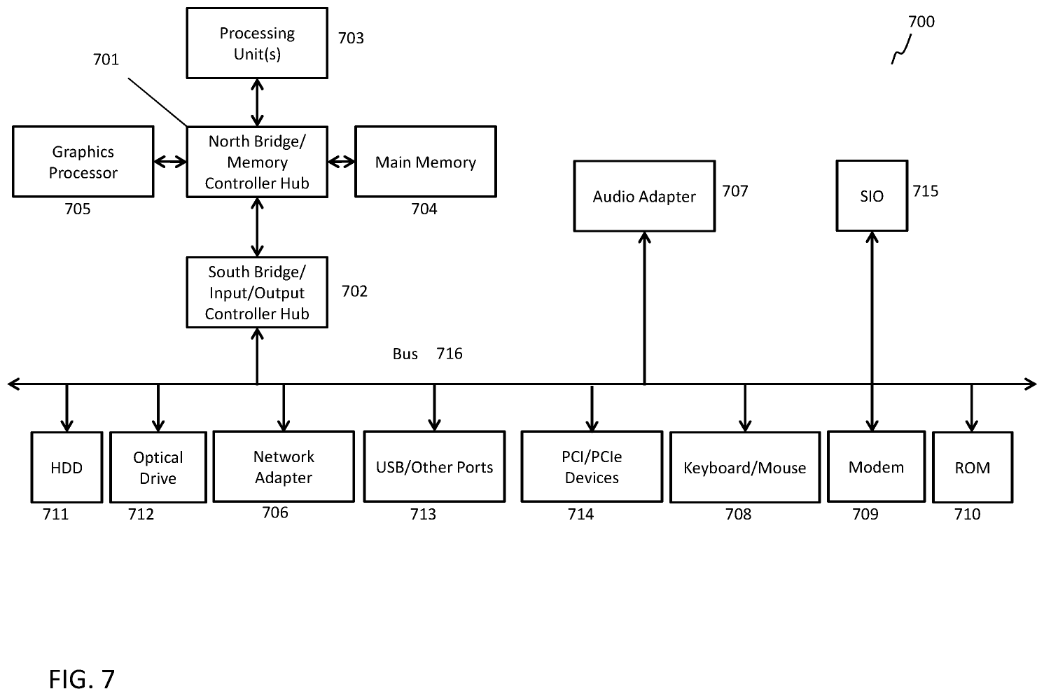

FIG. 7 is a block diagram of an example data processing system 700 in which aspects of the illustrative embodiments are implemented. Data processing system 700 is an example of a computer, such as a server or client device, in which computer usable code or instructions implementing the process for illustrative embodiments of the present invention are located. In one embodiment, FIG. 7 may represent a server computing device.

In the depicted example, data processing system 700 can employ a hub architecture including a north bridge and memory controller hub (NB/MCH) 701 and south bridge and input/output (I/O) controller hub (SB/ICH) 702. Processing unit 703, main memory 704, and graphics processor 705 can be connected to the NB/MCH 701. Graphics processor 705 can be connected to the NB/MCH 701 through, for example, an accelerated graphics port (AGP).

In the depicted example, a network adapter 706 connects to the SB/ICH 702. An audio adapter 707, keyboard and mouse adapter 708, modem 709, read only memory (ROM) 710, hard disk drive (HDD) 711, optical drive (e.g., CD or DVD) 712, universal serial bus (USB) ports and other communication ports 713, and PCI/PCIe devices 714 may connect to the SB/ICH 702 through bus system 716. PCI/PCIe devices 714 may include Ethernet adapters, add-in cards, and PC cards for notebook computers. ROM 710 may be, for example, a flash basic input/output system (BIOS). The HDD 711 and optical drive 712 can use an integrated drive electronics (IDE) or serial advanced technology attachment (SATA) interface. A super I/O (SIO) device 715 can be connected to the SB/ICH 702.

An operating system can run on processing unit 703. The operating system can coordinate and provide control of various components within the data processing system 700. As a client, the operating system can be a commercially available operating system. An object-oriented programming system, such as the Java™ programming system, may run in conjunction with the operating system and provide calls to the operating system from the object-oriented programs or applications executing on the data processing system 700. As a server, the data processing system 700 can be an IBM® eServer™ System p® running the Advanced Interactive Executive operating system or the Linux operating system. The data processing system 700 can be a symmetric multiprocessor (SMP) system that can include a plurality of processors in the processing unit 703. Alternatively, a single processor system may be employed.

Instructions for the operating system, the object-oriented programming system, and applications or programs are located on storage devices, such as the HDD 711, and are loaded into the main memory 704 for execution by the processing unit 703. The processes for embodiments described herein can be performed by the processing unit 703 using computer usable program code, which can be located in a memory such as, for example, main memory 604, ROM 710, or in one or more peripheral devices.

A bus system 716 can be comprised of one or more busses. The bus system 716 can be implemented using any type of communication fabric or architecture that can provide for a transfer of data between different components or devices attached to the fabric or architecture. A communication unit such as the modem 709 or the network adapter 706 can include one or more devices that can be used to transmit and receive data.

Those of ordinary skill in the art will appreciate that the hardware depicted in FIG. 7 may vary depending on the implementation. Other internal hardware or peripheral devices, such as flash memory, equivalent non-volatile memory, or optical disk drives may be used in addition to or in place of the hardware depicted. Moreover, the data processing system 700 can take the form of any of a number of different data processing systems, including but not limited to, client computing devices, server computing devices, tablet computers, laptop computers, telephone or other communication devices, personal digital assistants, and the like. Essentially, data processing system 700 can be any known or later developed data processing system without architectural limitation.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The system and processes of the figures are not exclusive. Other systems, processes, and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112(f) unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as they fall within the true spirit and scope of the invention.

What is claimed is:

1. A method of tracking a workout comprising:
receiving, from at least one user device, set-based exercise data associated with a user, wherein the set-based exercise data has a temporal component;
obtaining, using the processor, a plurality of characteristics of one or more known exercises;
identifying, based on the plurality of characteristics, that the set-based exercise data comprises at least one of the one or more known exercises;
analyzing, using the processor, the set-based exercise data to determine one or more workout metrics; and
storing, using the processor, the one or more workout metrics.

2. The method of claim 1, further comprising:
transforming the set-based exercise data using at least one of: a frequency-based transform or a feature-based transform; and
classifying, based on the transformed set-based exercise data and the plurality of characteristics, one or more portions of the set-based exercise data as one or more classified exercises.

3. The method of claim 2, further comprising:
selecting, based on the classifying, a portion of the plurality of characteristics specific to the one or more classified exercises; and
determining, based on the transformed set-based data, the one or more classified exercises, and the plurality of characteristics, a start time and an end time associated with at least one of the one or more classified exercises,
wherein analyzing the set-based exercise data further comprises analyzing only the set-based exercise data associated with the start time and the end time.

4. The method of claim 1, wherein the at least one user device is a device selected from the group consisting of: a wearable device, an optical sensing device, a motion sensing device, image based tracking device, a smartphone, augmented reality device, electromagnetic field sensors, and radio frequency sensors.

5. The method of claim 1, further comprising
obtaining, using the processor, an expected workout plan comprising at least one of: a workout type, a rep count, a weight amount, a workout duration, a workout location, a user ID, and historical workout data; and
selecting, based on the expected workout plan, a portion of the plurality of characteristics.

6. The method of claim 5, wherein the identifying further comprises:
transforming the set-based exercise data using at least one of: a frequency-based transform or a feature-based transform; and
determining, based on the transformed set-based data and the portion of the plurality of characteristics, a start time and an end time associated with at least one of the one or more known exercises.

7. The method of claim 1, wherein the workout metrics further comprise at least one of: a known exercise, rep count, rep duration, set count, set duration, pacing, form characteristics, exercise difficulty characteristics, range of motion, weight amount, rate of perceived exertion, estimated one rep max, number of reps a user could perform, exercise type, one or more user demographics, time of day, and historical workout metrics.

8. The method of claim 1, further comprising:
evaluating, using the processor, a portion of the one or more workout metrics; and
providing, based on the evaluation, at least one of: interpretable workout metrics and workout suggestions to at least one of: a user, a trainer, a coach, and an organization.

9. The method of claim 1, wherein the analyzing is performed in real time.

10. The method of claim 1, further comprising, storing, in a buffer storage, the received set-based exercise data, wherein the set-based exercise data stored in the buffer storage is analyzed after the completion of at least one of: a rep, a set, a workout, a plurality of workouts, and a time period.

11. A method of improving a workout comprising:
obtaining, using a processor, one or more workout metrics associated with known set-based exercise data;
evaluating, based on a plurality of statistical classification metrics, one or more workout metrics, wherein the statistical classification metrics comprise a plurality of characteristics associated with at least one of: exercise form and exercise difficulty;
assigning, based on the evaluation, a performance label to the one or more workout metrics, the performance label comprising at least one of a form label and an difficulty label; and
providing, using the processor, the performance label to an individual.

12. The method of claim 11, wherein the workout metrics comprises at least one of: a known exercise, rep count, rep duration, set count, set duration, pacing, form characteristics, exercise difficulty characteristics, range of motion, weight amount, rate of perceived exertion, estimated one rep max, number of reps a user could perform, exercise type, one or more user demographics, time of day, historical workout metrics, and the set-based exercise data.

13. The method of claim 11, wherein the individual is at least one of: a user, a trainer, a coach, and an organization.

14. The method of claim 11, wherein the evaluating involves transforming the workout metrics using at least one of: a frequency-based transform or a feature-based transform.

15. The method of claim 11, wherein the assigning further comprises assigning the performance label to at least one of a rep, a sequence of reps, and a set.

16. The method of claim 11, wherein the difficulty label is selected from at least one of: a percent of one repetition max, a number of repetitions remaining before failure, a rate of perceived exertion, and a descriptive identifier.

17. The method of claim 11, wherein the form label is selected from at least one of: a predefined set of exercise-specific form types, a numerical rating, and a descriptive identifier.

18. The method of claim 11, wherein the assigning is further based on historical workout metrics.

19. The method of claim 11, further comprising providing, based on the selected performance label, one or more workout suggestions to at least one of: a user, a trainer, a coach, and an organization.

20. A method comprising:
receiving, from at least one user device, set-based exercise data associated with a user, wherein the set-based exercise data has a temporal component;
obtaining, using a processor, a plurality of characteristics of one or more known exercises;
identifying, based on the plurality of characteristics, that the set-based exercise data comprises at least one of the one or more known exercises;
analyzing, using the processor, the set-based exercise data to determine one or more workout metrics;
evaluating, based on a plurality of statistical classification metrics, one or more workout metrics, wherein the statistical classification metrics comprise a plurality of characteristics associated with at least one of: exercise form and exercise difficulty;
assigning, based on the evaluation, a performance label to the one or more workout metrics, the performance label comprising at least one of a form label and an difficulty label;
providing, using the processor, at least one of: the workout metrics and the performance label to an individual; and
storing, using the processor, at least one of: the one or more workout metrics and the performance label.
